(19) United States
(12) Patent Application Publication
(10) Pub. No.: US 2007/0112042 A1
(43) Pub. Date: May 17, 2007

(54) Topical agent for application to the skin prior to luminous treatment

(76) Inventor: Flemming Kjaergaard Christensen, Hadsund (DK)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/652,611

(22) Filed: Jan. 12, 2007

Related U.S. Application Data
(62) Division of application No. 10/401,957, filed on Mar. 31, 2003.

(51) Int. Cl. A61K 31/455

(52) U.S. Cl. 514/356; 607/86

(57) ABSTRACT

There is disclosed a topical agent in the form of a topical vasodilator for the application to the skin prior to luminous treatment, e.g. laser treatment, wherein said topical vasodilator in a suitable vehicle includes the active substance consisting of nicotinic acid (Niacin) in the range of 0.5-10.0% by weight Preferably, the topical vasodilator is a skin cream containing the active substance, Niacin, in the range of 5% by weight.

### CROSS-REFERENCE TO RELATED APPLICATION

This application is a divisional application of us application serial no. 10/401,957, filed Mar. 31, 2003, the contents of which are incorporated herein by reference.

### BACKGROUND OF THE INVENTION

1. Field of the Invention

The present invention relates to a topical agent for application to the skin prior to luminous treatment.

2. Description of the Prior Art

Luminous treatment of the skin have been shown to enhance dermal collagen production by way of example by laser treatment by a pulsed dye laser during non-ablative wrinkle treatment by targeting chromophores (haemoglobin) in the microvasculature of the dermis. A mechanism is triggered which ultimately results in an increased production of collagen at the irradiated site.

### SUMMARY OF THE INVENTION

The invention provides a topical agent for application to the skin prior to luminous treatment in order to further enhance the dermal collagen production.

According to the present invention there is provided a topical agent in the form of a topical vasodilator for the application to the skin prior to luminous treatment, which topical vasodilator is in a suitable vehicle including an active substance consisting of nicotinic acid (Niacin) in the range of 0.5-10.0 % by weight.

Tests have been carried out for studying the dermal collagen production following topical application of Niacin prior to irradiation with a 585 nm pulsed dye laser. The effect of irradiation by the haemoglobin specific 585 nm pulsed dye laser is significantly increased by the application of topical Niacin at the proposed treatment site prior to treatment. This effect was supported with biochemical analysis which showed an increase in the PIIINP levels above that of irradiation alone. Therefore, a combination of light and topical preparations demonstrate improved efficiency for non-ablative skin rejuvenation. There were no adverse side-effects shown when irradiation was applied to study sites at sub-purpuric levels with fluences at 1.8 J/cm².

The topical vasodilator according to an embodiment of the invention is a skin cream containing 0.5 % by weight of the active substance, Niacin.

The topical vasodilator according to another embodiment of the invention is a skin cream containing 2.5 % by weight of the active substance, Niacin.

The topical vasodilator according to a further embodiment of the invention is a skin cream containing 5.0 % by weight of the active substance, Niacin.

The topical vasodilator according to a still further embodiment of the invention is a skin cream containing 7.5% by weight of the active substance, Niacin.

The topical vasodilator according to a still further embodiment of the invention is a skin cream containing 10.0 % by weight of the active substance, Niacin.

In the following the invention is explained in more details by means of a number of detailed examples of skin creams containing the active substance Niacin:

### EXAMPLE 1

A skin cream containing:

| | | |
|---|---|---|
| Carpopol ETD 2020 | 84.50% | (thickening agent) |
| Niacin | 0.50% | (nicotinic acid) |
| Lubragel DV | 14.35% | (viscous agent) |
| Diazolodial Urea | 0.25% | |
| Methylparaben | 0.25% | |
| Cetyl-alcohol | 0.15% | |

The pH-value is adjusted between 4 and 6

### EXAMPLE 2

| | | |
|---|---|---|
| Carpopol ETD 2020 | 40.00% | (thickening agent) |
| Niacin | 7.50% | (nicotinic acid) |
| Lubragel DV | 50.00% | (viscous agent) |
| Diazolodial Urea | 0.25% | |
| Methylparaben | 0.25% | |
| Cetyl-alcohol | 0.15% | |

The pH-value is adjusted between 4 and 6

As an alternative to the above-mentioned laser treatment by a 585 am pulsed dye laser it should be mentioned that the luminous treatment of the skin may be effected by irradiation treatment of the skin with a flashlight. However, in both cases the application of the topical vasodilator according to the present invention to the skin prior to the luminous treatment have shown to enhance dermal collagen production.

Finally it should be mentioned that the topical vasodilator according to the invention may be applied to the skin in the form of a solution, a lotion, a gel or a cream containing the active substance, Niacin.

What is claimed is:

1. A method for enhancing production of collagen by treatment of skin with a light source to provide skin rejuvenation comprising:
providing a topical agent including an active agent containing nicotinic acid (Niacin) in a range of 0.5 to 10% by weight of the topical agent;
applying the topical agent to an area of the skin to be rejuvenated in an amount sufficient to cause enlargement of blood vessels of the skin; and
applying light to the area of the skin sufficient to enhance collagen production at the area of the skin.

2. A method in accordance with claim 1 wherein:
the light is from a pulsed laser.

3. A method in accordance with claim 2 wherein:
the pulsed laser has a wavelength of 585 nm.

4. A method in accordance with claim 1 wherein:
the nicotinic acid ranges between 0.5% and 7.5% by weight of the topical agent.

5. A method in accordance with claim 2 wherein:
the nicotinic acid ranges between 0.5% and 7.5% by weight of the topical agent.

6. A method in accordance with claim 3 wherein:
the nicotinic acid ranges between 0.5% and 7.5% by weight of the topical agent.

7. A method in accordance with claim 1 wherein:
the nicotinic acid ranges between 0.5% and 5% by weight of the topical agent.

8. A method in accordance with claim 2 wherein:
the nicotinic acid ranges between 0.5% and 5% by weight of the topical agent.

9. A method in accordance with claim 3 wherein:
the nicotinic acid ranges between 0.5% and 5% by weight of the topical agent.

10. A method in accordance with claim 1 wherein:
the nicotinic acid ranges between 0.5% and 2.5% by weight of the topical agent.

11. A method in accordance with claim 2 wherein:
the nicotinic acid ranges between 0.5% and 2.5% by weight of the topical agent.

12. A method in accordance with claim 3 wherein:
the nicotinic acid ranges between 0.5% and 2.5% by weight of the topical agent.

13. A method in accordance with claim 1 wherein:
the nicotinic acid ranges between 2.5% and 10% by weight of the topical agent.

14. A method in accordance with claim 2 wherein:
the nicotinic acid ranges between 2.5% and 10% by weight of the topical agent.

15. A method in accordance with claim 3 wherein:
the nicotinic acid ranges between 2.5% and 10% by weight of the topical agent.

16. A method in accordance with claim 1 wherein:
the nicotinic acid ranges between 5% and 10% by weight of the topical agent.

17. A method in accordance with claim 2 wherein:
the nicotinic acid ranges between 5% and 10% by weight of the topical agent.

18. A method in accordance with claim 3 wherein:
the nicotinic acid ranges between 5% and 10% by weight of the topical agent.

19. A method in accordance with claim 1 wherein:
the nicotinic acid ranges between 7.5% and 10% by weight of the topical agent.

20. A method in accordance with claim 2 wherein:
nicotinic acid ranges between 7.5% and 10% by weight of the topical agent.

21. A method in accordance with claim 3 wherein:
nicotinic acid ranges between 7.5% and 10% by weight of the topical agent.
